(19) United States
(12) Patent Application Publication
(10) Pub. No.: US 2021/0186341 A1
(43) Pub. Date: Jun. 24, 2021

(54) PERSON SENSOR AND PERSON SENSOR SYSTEM

(71) Applicant: Drägerwerk AG & Co. KGaA, Lübeck (DE)

(72) Inventors: Roelof BERG, Lübeck (DE); Marke SPIESS, Hildesheim (DE); Jens LEMBKE, Groß Grönau (DE)

(21) Appl. No.: 17/057,956

(22) PCT Filed: Mar. 29, 2019
(86) PCT No.: PCT/EP2019/057973

§ 371 Date: Nov. 23, 2020

(30) Foreign Application Priority Data
May 24, 2018 (DE) 10 2018 004 202.3
Jul. 24, 2018 (DE) 10 2018 005 281.9

(51) Int. Cl. A61B 5/0205; G01D 5/12; G01B 7/00; A61B 5/00; A61B 5/242

(52) U.S. Cl. CPC A61B 5/02055; G01D 5/12; G01B 7/003; A61B 5/6804; A61B 5/0816; A61B 2562/0223; A61B 2503/04; A61B 2503/40; A61B 5/242

(57) ABSTRACT

A person sensor system (1) includes a person sensor (2) to determine personal parameters of a person, a holding device (3) for holding the person sensor (2) on an article of clothing of the person in interaction with the person sensor (2) and a magnetic device (4) with a first magnetic device section (5) and with a second magnetic device section (6) for attracting each other by magnetic force. The first magnetic device section (5) is arranged at the person sensor (2) and the second magnetic device section (6) is arranged at the holding device (3). A magnetic field detection device (7) detects a magnetic field of the magnetic device (4). The person sensor system (1) is configured to set an operating state of the person sensor system (1) as a function of the magnetic field detected. A person monitoring system (28) includes the person sensor system (1).

### CROSS REFERENCE TO RELATED APPLICATIONS

This application is a United States National Phase Application of International Application PCT/EP2019/057973, filed Mar. 29, 2019, and claims the benefit of priority under 35 U.S.C. § 119 of German Applications 10 2018 004 202.3, filed May 24, 2018 and 10 2018 005 281.9, filed Jul. 4, 2018, the entire contents of which are incorporated herein by reference.

### TECHNICAL FIELD

The present invention pertains to a person sensor system with a person sensor to determine personal parameters of a person, especially a patient, a baby, a toddler or the like. The person sensor system may be configured, for example, to monitor the personal parameters and/or to provide the personal parameters and/or the personal data generated from the personal parameters to a person monitoring system. The present invention further pertains to a person monitoring system with such a person sensor system.

### TECHNICAL BACKGROUND

Many different devices and systems for determining or monitoring such personal parameters as, for example, heart rate, body temperature, respiration or the like, of a person, especially of a patient, a baby, a toddler or the like are known. So-called person sensors are used to determine the personal parameters. A distinction is essentially made here between three different types of person sensors.

A first type of person sensor is configured to determine personal parameters by direct contact with the person. This type of person sensor includes, for example, heart rate sensors that are configured to measure the skin resistance of the person. A second type of person sensor is configured to determine personal parameters over a certain distance to the person of up to several meters. This type of person sensor includes, for example, body temperature sensors which are configured to measure a body temperature of the person by means of an infrared sensor. A third type of person sensor is configured to measure personal parameters from the direct surrounding area of the person. Such person sensors can be fixed, for example, to a fastening section of the person, for example, to an article of clothing, to a sensor holding device, to a dressing or the like. Prior-art person sensors have, for example, a clamping device, a holding device or the like for this purpose. Furthermore, prior-art person sensors have, for example, an acceleration sensor for measuring accelerations of the fastening section. Personal parameters of the person, for example, heart rate or respiration, can be easily derived via the measured accelerations. The third type of person sensor will, in particular, be taken into consideration within the framework of the present invention.

Person sensors according to the third type of person sensor are configured as fixable to the clothing of the person, as already explained above. Different fixing devices are known for this. A person sensor that can be arranged in a pocket formed on an article of clothing of a person is known from US 2015/0250419 A1. The pocket is configured as closable to secure the person sensor against sliding out of the pocket. For this purpose, the article of clothing has, for example, a fastening device with a fastening hook and with a fastening loop. As an alternative, flexible or fixed seams, a magnetic fastener or a snap fastener may be provided for closing the pocket. Such a mount for a person sensor has the drawback that a relatively large relative movement between the person sensor and the pocket is possible. Measuring inaccuracies may result from such a relative movement.

A magnetic fastening for fastening the person sensor to an article of clothing of the person is known from JP 11169208 A. A button-type permanent magnet can be brought to mesh with a pot-shaped magnetic counterholder in a positive-locking manner. Such a magnetic fastening has, in particular, the drawback that the separation of the permanent magnet and the counterholder is only possible with a high exertion of force. In this connection, a user can slip off or damage an article of clothing clamped between the permanent magnet and the counterholder. WO 2005/051311 A2 discloses a magnetic clamping device for a device for avoiding unintentional urination during sleep. The clamping device has a flexible strap, which is held at a basic unit on one side, with a magnetic end area facing away from the basic unit, which end area can be arranged at a permanent magnet that is arranged at the basic unit. In this state, the strap is arranged in a loop-shaped manner. An article of clothing can thus be clamped between the basic unit and the magnetic end area. Severing of the connection between the magnetic end area and the permanent magnet is made easier, for example, by a corresponding pulling on the strap. Nevertheless, the full magnetic force must be overcome for this purpose.

Prior-art fastening devices with permanent magnets especially have the drawback that the presence of a magnetic connection of a counterholder with the person sensor cannot be detected. In addition, a detachment of the person sensor from an article of clothing of the person can only be detected by the absence of the detection of personal parameters. The personal parameters determined in such a case correspond to the personal parameters received from a lifeless person, so that in this case no clear statement about the state of the person sensor or of the person to be monitored can be made.

### SUMMARY

Based on this state of the art, a basic object of the present invention is to provide a person sensor system, which does not have or at least partially does not have these drawbacks. Hence, the object of the present invention is to create a person sensor system, which guarantees in a simple and cost-effective manner an improved monitoring of an operating state of the person sensor system. The above object is accomplished by the present invention including a person sensor system with features according to the present invention and a person monitoring system with the features according to the present invention. Features and details which are described in connection with the person sensor system according to the present invention are, of course, valid in connection with the person monitoring system according to the present invention as well and vice versa, so that reference is and can always be made to the individual aspects of the present invention concerning the disclosure.

According to a first aspect of the present invention, the object is accomplished by a person sensor system. The person sensor system according to the present invention has a person sensor for determining personal parameters of a person, a holding device for holding the person sensor on an article of clothing of the person in interaction with the person sensor and a magnetic device with a first magnetic device section as well as with a second magnetic device section for attracting each other by magnetic force. The first magnetic device section is arranged at the person sensor and the second magnetic device section is arranged at the holding device. The person sensor system according to the present invention has a magnetic field detection device for detecting a magnetic field of the magnetic device, wherein the person sensor system is configured to set an operating state of the person sensor system as a function of the magnetic field detected.

Personal parameters are defined especially as parameters that can be determined at a person, which make it possible to infer a property of the person, especially a vital property, within the framework of the present invention. One possible personal parameter is, for example, a skin resistance, which changes as a function of the heart rate, so that the heart rate of the person can be determined by a detection of the skin resistance. Another personal parameter is a raising and lowering of the thorax or the diaphragm caused by the breathing of the person. By determining the raising and lowering, the respiration rate of the person can thus be determined. A temperature determined at the person, preferably on the skin of the person, can likewise be determined as a personal parameter. A body temperature can be determined from this. Blood pressure or other vital properties of the person can likewise be determined via suitable personal parameters.

The person to be monitored maybe, for example, a patient, a baby, a toddler or the like. Likewise, the monitoring of animals can be provided with a person sensor system according to the present invention. An article of clothing is also defined according to the present invention as a fastening device, which is especially made of a material that can be arranged on the body of the person, for example, by means of a strap, a loop, a belt or the like.

The person sensor system is configured for temporary fixing to the article of clothing of the person to be monitored. The person sensor system has the person sensor and the holding device for this purpose. The article of clothing can be arranged between the person sensor and the holding device and can be clamped by means of a clamping force acting between the person sensor and the holding device. The person sensor system has the magnetic device to generate the clamping force needed for clamping. The magnetic device has for this purpose the first magnetic device section arranged at the person sensor and the second magnetic device section arranged at the holding device. The first magnetic device section and the second magnetic device section are configured to attract each other by a magnetic force. For this reason, the first magnetic device section and/or the second magnetic device section have one or more magnets, especially neodymium magnets. The magnets may be arranged, for example, inside of or outside of the person sensor and of the holding device. Provisions may also be made for the magnetic device to form a partial area of a sensor housing of the person sensor and/or a partial area of a holding device housing of the holding device. In this case, for example, a magnet may be accessible from outside.

The magnetic field detection device is configured for detecting a magnetic field of the detection device. The magnetic field detection device is at least configured according to the present invention for detecting the magnetic field when the holding device is clamped at the person sensor and when an article of clothing is clamped between the holding device and the person sensor. In addition, the magnetic field detection device is configured for detecting no magnetic field or at least a changed magnetic field when the holding device has a greater distance or a defined relative position, for example, a relative rotation by 20°, 45° or 90° in relation to the person sensor. A greater distance is defined within the framework of the present invention as a distance, at which the clamping of the holding device and of the person sensor by means of the magnetic device is implausible. This distance may be, for example, 5 mm or more. The magnetic field detection device is preferably configured for detecting a magnetic field, which is generated only at a preferred position between the holding device and the person sensor. A preferred position is defined within the framework of the present invention especially as a relative position between the holding device and the person sensor, at which a clamping force has its maximum.

The magnetic field detection device may have, for example, a magnetic switch for detecting the magnetic field. The magnetic switch is preferably configured for switching a circuit when the holding device is arranged at the person sensor or the article of clothing is clamped between the holding device at the person sensor. In this manner, the magnetic field of this state can be determined by means of the magnetic field detection device. When the holding device is removed from the person sensor, the magnetic field is no longer sufficient for closing the magnetic switch from a defined distance, especially from 5 mm, or from a defined relative position, especially a relative rotation by 20° or more, so that the circuit is interrupted.

The person sensor is configured for determining the personal parameters of the person. The person sensor has for this purpose one or more sensors, in particular, an acceleration sensor, a temperature sensor or the like. In addition, the person sensor can be configured according to the present invention for analyzing the personal parameters, especially for determining personal data, e.g., heart rate, respiration rate, body temperature, blood pressure or the like. Provisions may be made for the person sensor to be configured for transmitting personal parameters and/or personal data to a person monitoring system, especially via a wireless connection. Furthermore, the person sensor may be configured to output a signal, especially an optical and/or acoustic signal or a vibration signal, as a function of the personal parameters determined. The person sensor preferably has a person sensor housing, which is especially made of a plastic, which protects an electronic system of the person sensor against external effects, for example, moisture, contamination or the like. The person sensor housing preferably has a smooth surface and is configured as easily cleanable in order to improve hygiene when using the person sensor system.

The person sensor system is configured to set the operating state of the person sensor system on the basis of the detected magnetic field, especially automatically. An automatic setting is defined within the framework of the present invention as the operating state being set or being able to be set without a further necessary interaction of a user. An operating state of the person sensor system is defined, for example, as a determination of personal parameters, a determination of personal data from the determined personal parameters, a transmission of the personal parameters and/or personal data and/or warning signals and/or the like to a person monitoring system, especially via a wireless connection, a direct outputting of a warning signal, especially of an optical and/or acoustic warning signal, a setting of a detection sensitivity during the detection of personal parameters and the like. The person sensor system is preferably configured to analyze the detected magnetic field by means of a variance comparison and thus to determine whether the holding device is arranged at the person sensor within predefined tolerances. In this way, it can be seen, for example, whether the holding device is especially in the preferred position, is arranged at the person sensor or is separated, for example, has fallen off, from same. The automatic setting of the operating state can accordingly comprise, for example, the outputting of a warning signal, especially at the person monitoring system. In this way, the falling off of the holding device can be signaled to a display device of the person monitoring system and/or via an acoustic alarm generator of the person monitoring system. In addition, the automatic setting of the operating state may comprise, for example, a switching off of the person sensor, especially to save electricity.

The person sensor system is preferably configured to set the operating state according to at least one of the following scenarios. In the case of a first scenario, the holding device is arranged in a starting position directly at the person sensor. It is detected by the person sensor system that no article of clothing is arranged between the person sensor and the holding device. When the holding device is subsequently removed from the person sensor, especially for fastening the person sensor system to an article of clothing, preferably no alarm is outputted because of the starting situation detected by the person sensor system. After the fastening of the person sensor system to the article of clothing, material is arranged between the person sensor and the holding device. This is detected by means of the person sensor system and automatically activates the operating state of the determination of the personal parameters as well as of the transmission of the personal parameters and the personal data to the person monitor. In this operating state, the person is monitored by means of the person sensor system.

In a second scenario, the person sensor system is arranged in the starting position on the article of clothing. This is determined by means of the person sensor system. Accordingly, the person is monitored in this operating state by means of the person sensor system. The holding device is then separated from the article of clothing by a user and is arranged directly at the person sensor. The separation is detected by the person sensor system. Therefore, it is preferred that the person sensor system has a predefined or settable inertia for outputting an alarm, so that an alarm is outputted only if the separation outlasts a predefined or adjustable period of time. If the bringing together of the person sensor and the holding device takes place—with or without material between them—within this time period, no alarm is outputted. The direct arrangement of the holding device at the person sensor is detected by the person sensor system. The alarm function can consequently be deactivated automatically, so that no alarm is outputted as well in case of a new separation of the holding device from the person sensor. Further, determination of the personal parameters as well as transmission of the personal parameters and of the personal data in this operating state are deactivated.

Compared to conventional person sensor systems, a person sensor system according to the present invention has the advantage that a configuration of the person sensor system can be determined with simple means as well as in a cost-effective manner and an operating state of the person sensor system can be set automatically as a function of the determined configuration. A falling off of the person sensor from the article of clothing of the person can thus be detected, and a corresponding warning message can be outputted to a user of the person sensor system. Likewise, a transmission power of the person sensor system can be controlled as a function of the determined configuration. Further, the person sensor and/or a wireless module of the person sensor system can, for example, be switched on and be switched off as a function of the determined configuration. The life or the operating time of a battery of the person sensor system can advantageously be extended hereby.

According to a preferred variant of the present invention, provisions may be made in the case of a person sensor system for the magnetic field detection device to be configured as a magnetometer for detecting a magnetic flux density of the magnetic device, wherein the person sensor system is configured to control the person sensor system as a function of the magnetic flux density detected. The magnetometer may be configured, for example, in addition, as a magnetometer of the sensor device to determine personal parameters. The magnetic field of the magnetic device has different magnetic flux densities as a function of the distances of the first magnetic device section to the second magnetic device section. These flux densities can be reliably detected by means of the magnetometer, so that statements about the distances between the person sensor and the holding device can be made by means of the person sensor system, especially of the person sensor, based on the detected magnetic flux densities. The person sensor system is preferably configured to control the person sensor system on the basis of the detected magnetic flux densities and thus on the basis of the distances between the person sensor and the holding device. The precision of the detection of the magnetic field can be markedly improved by means of a magnetometer. Especially if the person sensor already has a magnetometer for determining personal parameters, only minimal additional costs due to electronic components arise as a result.

It is preferable according to the present invention for the magnetic field detection device to be configured for determining a relative position and/or change in position of the holding device in relation to the person sensor. The magnetic field of the magnetic device has different magnetic flux densities as a function of the relative position of the first magnetic device section in relation to the second magnetic device section. These flux densities can be reliably detected by means of the magnetometer, so that statements about the relative positions between the person sensor and the holding device can be made by means of the person sensor system, especially of the person sensor, based on the detected magnetic flux densities. In this manner, a sliding off and/or a rotation of the holding device from the preferred position can already be reliably detected. The person sensor system is configured to control the person sensor system on the basis of the detected magnetic flux densities and thus on the basis of the relative positions between the person sensor and the holding device. In this manner, for example, a relative rotation, i.e., a rotary relative movement, or shifting, i.e., a translatory relative movement, of the person sensor and the holding device can be determined. The precision of the detection of the magnetic field can be further improved by means of such a magnetometer.

Moreover, the person sensor is preferably configured to automatically set an operating state of the person sensor system as a function of the determined relative position and/or change in position of the holding device in relation to the person sensor. The determined relative movements can be interpreted accordingly by the person sensor system, especially by the person sensor, as a control command for the person sensor, for example, as switching on, changing the sensitivity of the person sensor, changing of a loudness or the like. In this way, the operating comfort of the person sensor can be improved considerably. In addition, the number of operating elements of the person sensor can hereby be reduced.

In an especially preferred embodiment of the present invention, provisions may be made in a person sensor system for the magnetic field detection device to be arranged at the person sensor. The magnetic field detection device is preferably arranged within a sensor housing of the person sensor. The person sensor has a large installation space in relation to the holding device. An integration of the magnetic field detection device into the person sensor is thus possible with simple means as well as in a cost-effective manner.

The first magnetic device section preferably has a first magnet, in particular a neodymium magnet. Neodymium magnets are characterized by an especially high magnetic flux density and a relatively minimal space needed for installation. An integration of a magnet into the person sensor is possible in a cost-effective manner as well as with simple means because of the relatively large installation space available.

Furthermore, the second magnetic device section preferably has a ferromagnetic element. Without providing a magnetic flux itself, a ferromagnetic element can be attracted by a magnet. Further, a magnetic field of the magnet of the first magnetic device section can be influenced or be detuned by means of the ferromagnetic element such that this change can be detected by means of the magnetic field detection device. The ferromagnetic element contains, for example, iron and/or nickel and/or cobalt. The ferromagnetic element is preferably arranged at the holding device, and the magnet is preferably arranged at the person sensor such that the holding device can be moved into a preferred position in relation to the person sensor due to the magnetic attraction of the first magnetic device section and of the second magnetic device section. A ferromagnetic element has the advantage that it can be easily integrated into a relatively limited installation space of the holding device. It can, in addition, be manufactured in a cost-effective manner.

According to a preferred embodiment of the present invention, the second magnetic device section has a magnet, in particular a neodymium magnet. An additional magnet has the advantage that an especially major effect can be exerted on the magnetic field due to the arrangement of the holding device at the person sensor. A reliability of the detection of the magnetic field by means of the magnetic field detection device is thus improved with simple means as well as in a cost-effective manner. Moreover, a clamping force for clamping the article of clothing can be improved in this manner, so that the risk of an unintentional falling off of the person sensor is reduced.

The magnet of the first magnetic device section and the magnet of the second magnetic device section are especially preferably arranged and oriented such that a preferred position can hereby be brought about between the person sensor and the holding device. This has the advantage that an arrangement of the person sensor system on an article of clothing of a person is improved. Further, it can consequently be guaranteed that after a deliberate movement of the person sensor from the preferred position by a user, especially for the input of a control command at the person sensor system, an automatic movement back into the preferred position is ensured.

The magnet of the first magnetic device section and the magnet of the second magnetic device section are preferably arranged and oriented such that the first magnetic device section and the second magnetic device section attract each other in a first relative position from the person sensor and the holding device and repel each other in a second relative position from the person sensor and the holding device, which second relative position is different from the first relative position. In the first relative position, at least one permanent magnet of the person sensor and at least one permanent magnet of the holding device attract each other. A plurality of permanent magnets of the person sensor and of the holding device preferably attract each other in the first relative position. The permanent magnets attracting each other in the first relative position are preferably directly adjacent to one another. The second relative position is different from the first relative position. The second relative position can preferably be reached by a rotation of the holding device about an axis. In the second relative position between the holding device and the person sensor, at least one permanent magnet of the first magnetic device section and at least one permanent magnet of the second magnetic device section repel each other. A plurality of permanent magnets of the first magnetic device section and of the second magnetic device section preferably repel each other in the second relative position. The permanent magnets repelling each other in the second relative position are preferably directly adjacent to one another. This has the advantage that a secure arrangement of the person sensor system at a fastening section, for example, on an article of clothing, on a dressing, on a sensor holding device or the like, of a person, for example, of a patient, and an easy removal of the person sensor system from the fastening section are guaranteed with simple means as well as in a cost-effective manner. Due to the attraction to each other of the first magnetic device section and the second magnetic device section in the first relative position of the person sensor and of the holding device, an especially firm hold of the person sensor system at the fastening section is achieved. The risk of an unwanted slipping or detachment of the person sensor system from the fastening section is thus considerably reduced. Due to the first magnetic device section and the second magnetic device section repelling each other in the second relative position of the person sensor and the holding device, an especially easy detachment of the holding device from the person sensor is guaranteed. The movement of the holding device or of the person sensor from the first relative position into the second relative position is easily possible with simple means. A person sensor system fastened to the fastening section can thus be easily detached from same. In addition, a relative alignment of the person sensor and the holding device is improved by means of such a magnetic device because of the attracting as well as the repelling magnetic forces.

Further, the first magnetic device section preferably has at least two magnets which are arranged at a spaced location from one another, wherein the at least two magnets are oriented such that different magnetic poles of the magnets are facing towards an interior of the person sensor. Two magnets are preferably spaced apart by 180° from one another and have the same distance to the axis in relation to a surface center or an axis for rotating the holding device from the first relative position into the second relative position. Further, the magnets of the second magnetic device section are preferably arranged correspondingly, so that the at least two magnets of the holding device in the first relative position are each assigned to one of the magnets of the person sensor, wherein each of these pairs of magnets is arranged opposite with different magnetic poles, so that the respective magnets of a pair of magnets attract each other. In the second relative position, which is reached after a rotation of the holding device by 180° about the axis, the magnets of the pair of magnets are transposed, so that now each of these magnet pairs with identical magnetic poles repel each other. An especially good hold of the holding device at the person sensor and an especially easy separation of the holding device from the person sensor are guaranteed with simple means as well as in a cost-effective manner by means of such a first magnetic device section and a second magnetic device section.

According to a preferred embodiment, the first magnetic device section has at least four magnets which are arranged at a spaced location from one another, wherein the at least four magnets are oriented such that alternately different magnetic poles are facing an interior of the person sensor. The magnets are preferably spaced apart by 90° from one another and have the same distance to the axis in relation to a surface center or an axis for rotating the holding device from the first relative position into the second relative position. Further, the second magnetic device section is preferably configured such that the at least four magnets of the holding device in the first relative position are each assigned to one of the magnets of the person sensor, wherein each of these pairs of magnets is arranged opposite with different magnetic poles, so that the respective magnets of a pair of magnets attract each other. In the second relative position, which is reached after a rotation of the holding device by 90° into a desired direction about the axis, the magnets of the magnet pair are transposed, so that now each of these magnet pairs is arranged opposite with identical magnetic poles and the respective magnets of a magnet pair repel each other. An especially good hold of the holding device at the person sensor and an especially easy separation of the holding device from the person sensor are guaranteed with simple means as well as in a cost-effective manner by means of such a first magnetic device section and a second magnetic device section.

According to a preferred variant of the present invention, provisions may be made for the holding device to have a contact area with a first base and the person sensor to have a countercontact area with a second base for arrangement at the contact area. A contact area is defined as an area of the holding device, which contacts the countercontact area of the person sensor in the first relative position. The first base and the second base preferably have a flat configuration. As an alternative, the first base and the second base have one or more raised areas or recessions, curvatures or the like, which are complementary to one another. The first base and/or the second base preferably have a round configuration. Permanent magnets of the magnetic device, which are assigned to attract each other and repel each other, preferably have the same distance to a surface center of the first base and of the second base. Such a person sensor system has the advantage that the holding device has a common contact area with the person sensor at least in the first relative position. Consequently, a hold of the holding device is improved with simple means as well as in a cost-effective manner.

It is especially preferred that the contact area have a positive-locking section and the countercontact area have a counter-positive-locking section which can be brought to mesh with the positive-locking section in the first relative position. The positive-locking section and the counter-positive-locking section preferably have complementary configurations, especially such that the counter-positive-locking section is configured as an inversion of the positive-locking section. The positive-locking section and/or the counter-positive-locking section preferably have a partly spherical, cylindrical or wedge-shaped configuration. The person sensor and the holding device can be easily arranged in a defined relative position to one another by means of the positive-locking section and of the counter-positive-locking section. The number of degrees of freedom of a relative movement of the person sensor and of the holding device, which is limited by the positive-locking section and by the counter-positive-locking section, is almost freely selectable. The positive-locking section and the counter-positive-locking section are preferably configured to allow only two degrees of freedom, namely a rotation about an axis and a translation along this axis. This has the advantage that the person sensor and the holding device can be easily brought into the first relative position as well as into the second relative position.

Provisions may be made according to the present invention for the contact area to have a shoulder relative to an adjacent section of the patient sensor and/or the countercontact area to have a shoulder relative to an adjacent section of the holding device. The shoulder is preferably configured as a projection, stop edge, linear guide shoulder or the like. A shoulder may especially be used as a stop surface for the person sensor or the holding device, so that a specific arrangement of the holding device at the person sensor is improved.

The shoulder preferably has a circumferential configuration. As a result, a recess, cup, depression or the like is preferably formed in the person sensor or in the holding device. A circumferential shoulder has the advantage that a relative alignment of the person sensor and the holding device can be improved. In addition, an unintentional sliding off of the holding device from the person sensor during the arrangement of the holding device at the person sensor can thus be avoided with simple means as well as in a cost-effective manner.

In addition, it is preferred that the shoulder have a circumferentially graduating configuration. Within the framework of the present invention, a circumferentially graduating shoulder is defined as a shoulder, which has different shoulder heights along a section. The shoulder preferably has a circumferentially linearly graduating configuration, so that a change in the shoulder height is constant. The shoulder is thus preferably configured according to a ramp or the like, so that lateral sliding off of the holding device at the person sensor is improved. In addition, an end stop is provided by the shoulder when the holding device is pushed onto the person sensor. A shoulder configured in this manner has the advantage that approaching of the holding device and the person sensor as well as reaching the first relative position are improved with simple means as well as in a cost-effective manner.

Further, the holding device can preferably be arranged at least partially in a positive-locking manner in a mount of the person sensor. The holding device can especially preferably be arranged entirely in a positive-locking manner in the mount of the person sensor. For this purpose, the person sensor has a recess, into which the holding device can be inserted.

The holding device can preferably be inserted into the recess such that this holding device does not protrude on the side over the person sensor and is arranged entirely within the mount. Compared to conventional person sensor systems, a person sensor system configured in this manner has the advantage that edges of the holding device can be retracted in the mount. In addition, the lateral sliding off of the holding device in relation to the person sensor is prevented due to the positive-locking mount. Such a configuration has the additional advantage that a setting of the first relative position and of the second relative position of the person sensor and the holding device is improved with simple means as well as in a cost-effective manner. For this purpose, the holding device is to be rotated relative to the person sensor, for example, only from the first relative position into the second relative position.

The holding device preferably has a grip surface, which projects from the holding device as well as faces away from the person sensor, wherein the grip surface is configured for manual introduction of a torque into the holding device. The grip surface preferably has a shape and size, which offer sufficient hold for the thumb and index finger or thumb and index finger tips of an operator of the person sensor to overcome a magnetic holding force exerted in the first relative position and to rotate the holding body into the second relative position. Hence, the grip surface is configured to receive the torque necessary for this purpose from the operator and to transfer same to the holding device. In the second relative position, the magnetic holding force is converted into a magnetic repulsion force, so that the holding device can be easily pulled off from the person sensor by gripping or holding the grip surface. A grip surface has the advantage that a movement of the holding device from the first relative position into the second relative position in relation to the person sensor is improved with simple means as well as in a cost-effective manner.

It is preferred according to the present invention that at least one partial area of the first magnetic device section is arranged in a central sensor rotation point (vertical sensor axis) of the person sensor and at least one partial area of the second magnetic device section is arranged in a central holding device rotation point (vertical axis of the holding device) of the holding device. In the preferred position, the sensor rotation point is adjacent to the holding device rotation point. It is ensured in this manner that the sensor rotation point is adjacent to the holding device rotation point even in case of a relative rotation of the person sensor and the holding device out of the preferred position. The sensor rotation point and the holding device rotation point are located on a rotation axis of this relative rotation. An unintentional lateral sliding off of the person sensor from the holding device can thus be prevented with simple means as well as in a cost-effective manner.

Further, the holding device preferably has a holding device projection at the holding device rotation point and the person sensor has a sensor mount at the sensor rotation point, wherein the sensor mount is configured for the at least partial mounting of the holding device projection. In addition, the holding device projection and the sensor mount are preferably configured to favor a relative rotation of the person sensor and the holding device, especially by means of smooth surfaces and/or a rotationally symmetrical geometry. A rotation axis, which offers an improved guiding, especially obliquely to the rotation axis, in case of the relative rotation of the person sensor and the holding device, is thus provided with simple means as well as in a cost-effective manner. The risk of the unintentional separation of the person sensor and the holding device in case of the relative rotation by the user is thus reduced.

The person sensor preferably has at least one electrically conductive person sensor contact surface and the holding device has at least one electrically conductive holding device contact surface, wherein the at least one person sensor contact surface and the at least one holding device contact surface are configured to close a common electrical circuit by direct mutual contact, wherein the person sensor system is configured to control the person sensor system as a function of the state of the circuit. The person sensor contact surface and the holding device contact surface are preferably arranged and configured such that the circuit is closed if the holding device is held in the preferred position at the person sensor. Accordingly, the circuit is interrupted by an article of clothing that is located between the holding device and the person sensor and configured as an electrical insulator. The person sensor is preferably configured to control the person sensor system, and especially the person sensor, as a function of the state of the circuit. The person sensor system is thus configured to control the person sensor system as a function of the determined magnetic field and the state of the circuit determined. In this case, a plurality of scenarios arise which are described in a simplified manner below. If a DESIRED magnetic field as well as a closed circuit are detected, the person sensor system can infer that the holding device is arranged directly at the person sensor. This may be, for example, an operating configuration of the person sensor system for a charging operation of the battery. If a DESIRED magnetic field as well as an open circuit are detected, the person sensor system can infer that an article of clothing is clamped between the holding device and the person sensor. This is the normal operating configuration of the person sensor system. If no DESIRED magnetic field as well as an open circuit are detected, the person sensor system can infer that the holding device and the person sensor are separated from one another, for example, when the person sensor has fallen off from the article of clothing because of a movement of the person. This has the advantage that a state or a configuration of the person sensor system can be better determined.

It is preferred that the circuit extends through the first magnetic device section and/or through the second magnetic device section. The first magnetic device section and/or second magnetic device section are thus integrated into the circuit. This has the advantage that a closing of the circuit can be improved because of the magnetic force.

Further, the first magnetic device section preferably has the person sensor contact surface and/or the second magnetic device section has the holding device contact surface. It is preferred in this case that a magnet or ferromagnetic element of the first magnetic device section be configured as a person sensor contact surface and/or a magnet or ferromagnetic element of the second magnetic device section be configured as a holding device contact surface. This has the advantage that the number of necessary components of the person sensor system can be reduced in this manner. An assembly effort as well as the manufacturing costs of the person sensor system can thus likewise be advantageously reduced.

According to a second aspect of the present invention, the object is accomplished by means of a person monitoring system for monitoring personal parameters of a person. The person monitoring system has a person sensor system with a person sensor for measuring at least one personal parameter of the person and a person monitor for outputting personal information based on the at least one personal parameter. According to the present invention, the person sensor system is configured as a person sensor system according to the first aspect of the present invention.

The same advantages that were already described in regard to a person sensor system according to the first aspect of the present invention are obtained in the person monitoring system according to the present invention. Accordingly, compared to the conventional person monitoring systems, the person monitoring system according to the present invention has the advantage that a configuration of the person sensor system can be determined with simple means as well as in a cost-effective manner and an operating state of the person system can be automatically set as a function of the determined configuration. Falling off of the person sensor from the article of clothing of the person can thus be detected, and a corresponding warning message can be outputted to a user of the person sensor system. Likewise, a transmission power of the person sensor system can be controlled as a function of the determined configuration. Further, the person sensor and/or a wireless module of the person sensor system can, for example, be switched on and be switched off as a function of the determined configuration. The life or the operating time of a battery of the person sensor system can advantageously be extended hereby.

Further measures improving the present invention appear from the following description of some exemplary embodiments of the present invention, which are shown in the figures. All the features and/or advantages, including design details and arrangements in space, which appear from the claims, from the description or from the drawings, may be essential for the present invention both in themselves and in the different combinations. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view of a preferred first embodiment of a person sensor system according to the present invention;

FIG. 2 is a side view of a preferred second embodiment of a person sensor system according to the present invention;

FIG. 3 is a top view of the preferred first embodiment of the person sensor system according to FIG. 1;

FIG. 4 is a top view of a preferred third embodiment of a person sensor system according to the present invention;

FIG. 5 is a side view of a preferred fourth embodiment of a person sensor system according to the present invention;

FIG. 6 is a side view of a preferred fifth embodiment of a person sensor system according to the present invention;

FIG. 7 is a top view of a preferred sixth embodiment of a person sensor system according to the present invention;

FIG. 8 is a side view of a preferred seventh embodiment of the person sensor system according to the present invention in the first relative position;

FIG. 9 is a side view of the preferred seventh embodiment of the person sensor system according to the present invention in the second relative position;

FIG. 10 is a side view of a preferred eighth embodiment of a person sensor system according to the present invention; and

FIG. 11 is a side view of a preferred embodiment of a person monitoring system according to the present invention.

### DESCRIPTION OF PREFERRED EMBODIMENTS

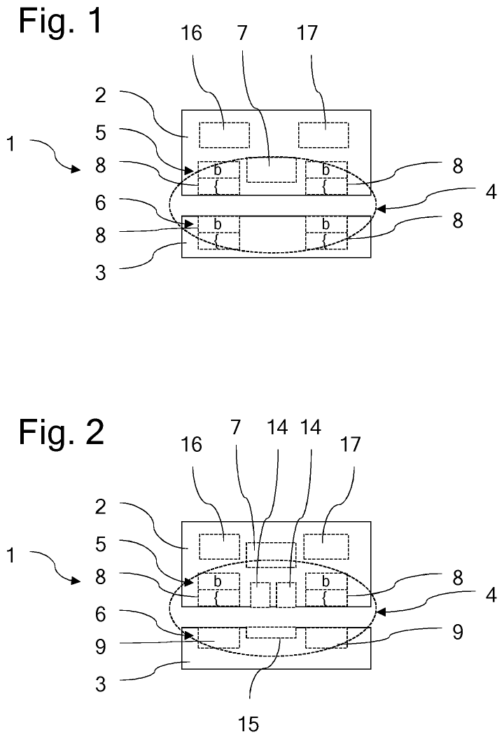

Referring to the drawings, elements having the same function and mode of operation are always designated by the same reference numbers in FIGS. 1 through 11.

FIG. 1 schematically shows a preferred first embodiment of a person sensor system 1 according to the present invention in a side view. The person sensor system 1 has a person sensor 2 and a holding device 3. In this example, the person sensor 2 and the holding device 3 have a cylindrical configuration. According to the present invention, the person sensor 2 and the holding device 3 may have almost any geometries, wherein a geometry of the holding device 3 is preferably adapted to a geometry of the person sensor 2. The sensor device, the person sensor 2, may have, for example, a sensor recess, not shown, into which the holding device 3 can be completely or at least partially mounted.

Further, the person sensor system 1 has a magnetic device 4 with a first magnetic device section 5 arranged at the person sensor 2 and with a second magnetic device section 6 arranged at the holding device 3. The first magnetic device section 5 and the second magnetic device section 6 have each a plurality of magnets 8 that are arranged at a spaced location from one another. The magnets 8 are arranged such that a preferred position between the person sensor 2 and the holding device 3 can be reached via the magnets 8. A magnetic field detection device 7 is arranged at the person sensor 2 to detect the magnetic field of the magnetic device 4. Further, the person sensor 2 has a transmission device 16 for the wireless transmission of data as well as a battery 17 for power supply.

FIG. 2 schematically shows in a side view a preferred second embodiment of a person sensor system 1 according to the present invention. The person sensor system 1 has a person sensor 2 and a holding device 3. In this example, the person sensor 2 and the holding device 3 have a cylindrical configuration. According to the present invention, the person sensor 2 and the holding device 3 may have almost any geometries, wherein a geometry of the holding device 3 is preferably adapted to a geometry of the person sensor 2. The person sensor 2 may have, for example, a sensor recess, not shown, into which the holding device 3 can be completely or at least partially mounted.

Moreover, the person sensor system 1 has a magnetic device 4 with a first magnetic device section 5 arranged at the person sensor 2 and with a second magnetic device section 6 arranged at the holding device 3. The first magnetic device section 5 has a plurality of magnets 8 arranged at a spaced location from one another. The second magnetic device section 6 has a plurality of ferromagnetic elements 9 arranged at a spaced location from one another. The magnets 8 and the ferromagnetic elements 9 are arranged such that a preferred position between the person sensor 2 and the holding device 3 can be reached via the magnets 8 and the ferromagnetic elements 9. A magnetic field detection device 7 is arranged at the person sensor 2 for detecting the magnetic field of the magnetic device 4. Furthermore, the person sensor 2 has two person sensor contact surfaces 14 and the holding device has a holding device contact surface 15. The person sensor contact surfaces 14 and the holding device contact surfaces 15 are arranged and configured to close a circuit with each other, when the holding device 3 is arranged in the preferred position directly at the person sensor 2. Further, the person sensor 2 has a transmission device for the wireless transmission of data as well as a battery 17 for power supply.

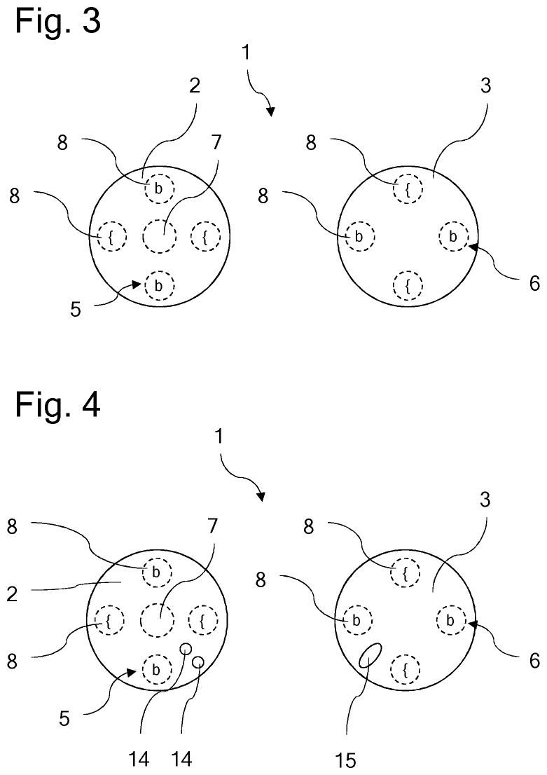

FIG. 3 schematically shows the preferred first embodiment of the person sensor system according to the present invention from FIG. 1 in a top view. Each of the sides of the person sensor 2 and of the holding device 3, which are located opposite one another in the preferred position, i.e., facing one another, can be seen. It can be seen in this view that the first magnetic device section 5 of the person sensor 2 and the second magnetic device section 6 of the holding device 3 have each four magnets 8 arranged at a spaced location from one another, wherein the magnets 8 are arranged in the circumferential direction with alternating polarity. Thus, a magnetic holding force has its maximum in the preferred position and a repulsion force between the first magnetic device section 5 and the second magnetic device section 6 has its maximum in a release position rotated by 90° in relation to the preferred position. Further, the magnetic field detection device 7 arranged at the person sensor 2 for detecting the magnetic field of the magnetic device 4 (cf. FIG. 1) can be seen.

FIG. 4 schematically shows a preferred third embodiment of a person sensor system 1 according to the present invention in a top view. Each of the sides of the person sensor 2 and of the holding device 3, which are located opposite one another, i.e., facing one another, in the preferred position, can be seen. The person sensor system 1 according to the preferred third embodiment differs from the person sensor system 1 according to the preferred second embodiment from FIG. 2 in the feature that the second magnetic device section 6 has magnets 8 instead of ferromagnetic elements 9 (cf. FIG. 2).

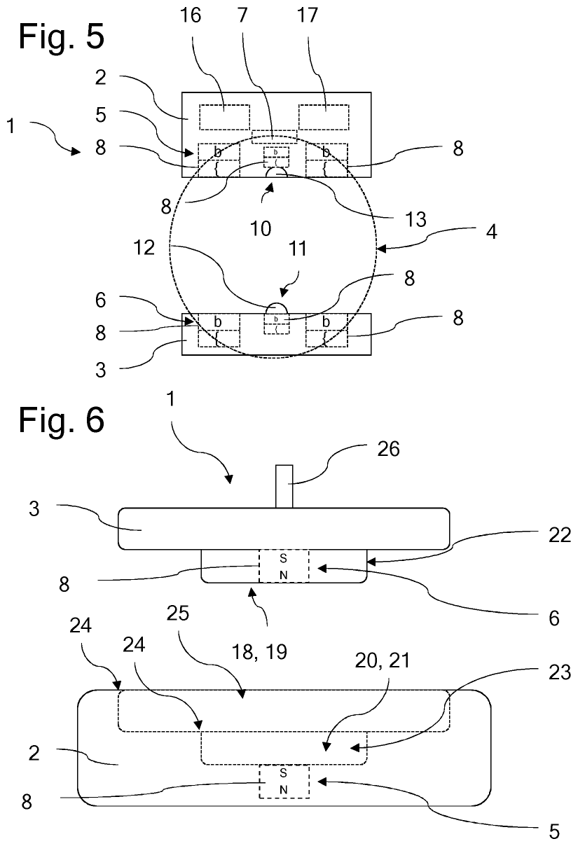

FIG. 5 schematically illustrates in a side view a preferred fourth embodiment of a person sensor system 1 according to the present invention. The fourth embodiment of the person sensor system 1 according to the present invention shown in FIG. 5 differs from the first embodiment of the person sensor system 1 according to the present invention shown in FIG. 1 in the features that the person sensor 2 in the area of a sensor rotation point 10 and the holding device 3 in the area of a holding device rotation point 11 has each a magnet 8, which attract each other in the preferred position. In addition, the person sensor 2 has a semispherical sensor mount 13 at the sensor rotation point 10 and the holding device 3 has a semispherical holding device projection 12 at the holding device rotation point 11. The sensor mount 13 is configured to mount the holding device projection 12. As a result, a relative rotation of the person sensor in relation to the holding device 3 is improved. Further, arrangement of the person sensor 2 and the holding device 3 is made easier for a user, since the sensor mount 13 and the holding device projection 12 may be used as orientation.

FIG. 6 schematically shows a preferred fifth embodiment of a person sensor system 1 according to the present invention in a side view. In this preferred fifth embodiment, the person sensor 2 has a mount 25 with a counter-positive-locking section 23 for the positive-locking mount of a positive-locking section 22 of the holding device 3. The mount 25 has two shoulders 24 formed around the mount 25. The holding device 3 can be arranged in the mount 25 such that only a grip surface 26 protrudes from the mount 25 for holding the holding device 3. According to an alternative embodiment, not shown, the holding device 3 may be arranged entirely within the mount 25.

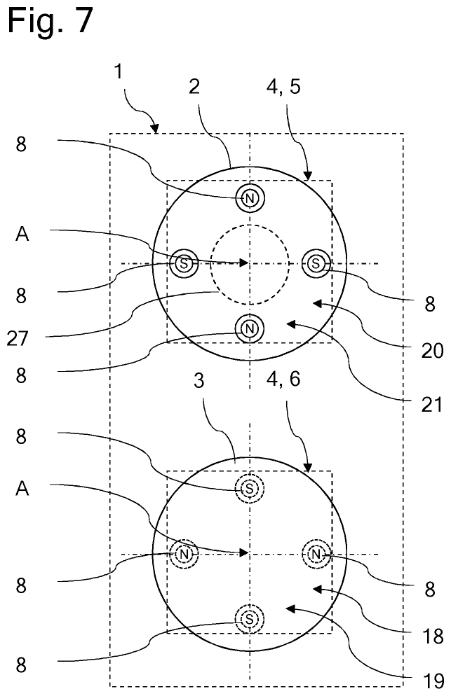

FIG. 7 schematically shows a preferred sixth embodiment of a person sensor system 1 according to the present invention in a top view. The first magnetic device section 5 of the magnetic device 4 has four magnets 8, which are distributed at a distance of 90° each on a circle about a central axis A of the person sensor 2, wherein the four magnets 8 are oriented such that alternatingly different magnetic poles face an interior of the person sensor 2. The second magnetic device section 6 of the magnetic device 4 also has four magnets 8 which are distributed at a distance of 90° each on a circle about a central axis A of the holding device 3, wherein the four magnets 8 are oriented such that alternatingly different magnetic poles face an interior of the holding device 3.

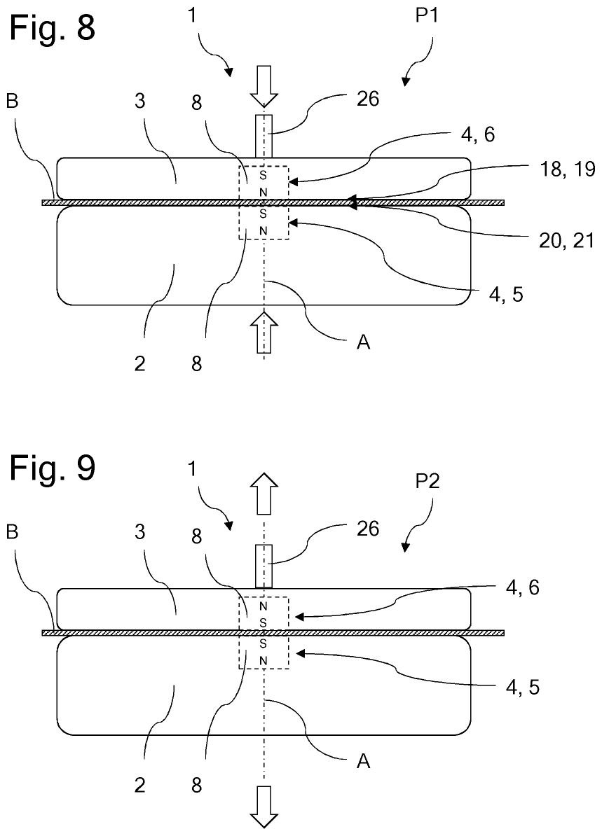

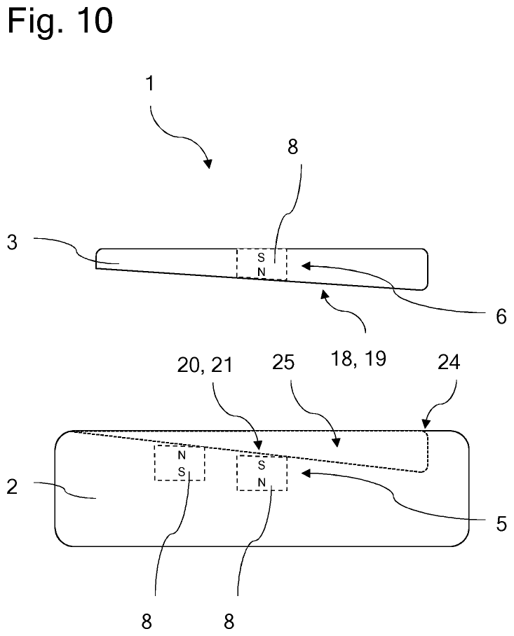

The holding device 3 can be arranged in a first relative position P1 (cf. FIG. 8) in relation to the person sensor 2 such that the four magnets 8 of the second magnetic device section 6 are located each opposite an opposite magnetic pole of a magnet 8 of the first magnetic device section 5, so that the first magnetic device section 5 and the second magnetic device section 6 attract each other. In this state, the person sensor system 1 may be fixed, for example, to a fastening section B (cf. FIG. 8) of a person, especially to an article of clothing, to a dressing, to a sensor holding device or the like. By means of rotation about the axis A by 90° in any desired direction, the holding device 3 can be brought from the first relative position P1 into a second relative position P2 (cf. FIG. 9) in relation to the person sensor 2 such that the four magnets 8 of the second magnetic device section 6 are each located opposite an identical magnetic pole of a magnet 8 of the first magnetic device section 5, so that the first magnetic device section 5 and the second magnetic device section 6 of the magnetic device 4 repel each other. In this state, the person sensor system 1 can be removed from the fastening section B of a person or the like.

FIG. 8 shows a preferred seventh embodiment of the person sensor system 1 according to the present invention in the first relative position P1 in a side view. In this example, the first magnetic device section 5 of the magnetic device 4 has, for example, two magnets 8, which have the same distance from the axis A and are spaced apart by 180° from one another. Correspondingly, the second magnetic device section 6 of the magnetic device 4 has two magnets 20, which have the same distance from the axis A as the two magnets 8 of the first magnetic device section 5 and are spaced apart by 180° from one another. In the first relative position P1, the magnetic poles of the magnets 8 of the first magnetic device section 5 are arranged located opposite opposed magnetic poles of the magnets 8 of the second magnetic device section 6, so that the first magnetic device section 5 and the second magnetic device section 6 attract each other. A fastening section B, for example, an article of clothing or the like, of a person is thus clamped between the countercontact area 20 of the person sensor 2 and the contact area 18 of the holding device 3.

FIG. 9 shows the preferred seventh embodiment of the person sensor system 1 according to the present invention from FIG. 8 in the second relative position P2 in a side view. In the second relative position P2, the magnetic poles of the magnets 8 of the first magnetic device section 5 are arranged located opposite identical magnetic poles of the magnets 8 of the second magnetic device section 6, so that the first magnetic device section 5 and the second magnetic device section 6 repel each other. In this state, the holding device 3 and the person sensor 2 can be easily removed from the fastening section B or the like of the person. In order to improve or facilitate a relative rotation of the holding device 3 in relation to the person sensor 2 from the first relative position P1 to the second relative position P2, a grip surface 26 is formed at the holding device 3, which grip surface 26 extends away from the person sensor 2. A user of the person sensor system 1 can better transmit a torque from his fingers to the holding device 3 via the grip surface 26.

FIG. 10 schematically shows in a side view a preferred eighth embodiment of a person sensor system 1 according to the present invention. The person sensor 2 has a mount 25 which is defined laterally by a graduating shoulder 24. Correspondingly, the holding device 3 has a wedge-shaped cross section and can be arranged entirely within the mount 25. In the inserted state, the person sensor 2 is arranged in the first relative position P1, and a magnet 8 of the holding device 3 is assigned to an opposed magnetic pole of a magnet 8 of the person sensor 2, so that these magnets 8 attract each other. To release the holding device 3 from the person sensor 2, the holding device 3 can be displaced along the countercontact area 20 into the second relative position P2, in which the magnet 8 of the holding device 3 is assigned to an identical magnetic pole of another magnet 8 of the person sensor 2, so that these magnets 8 repel each other.

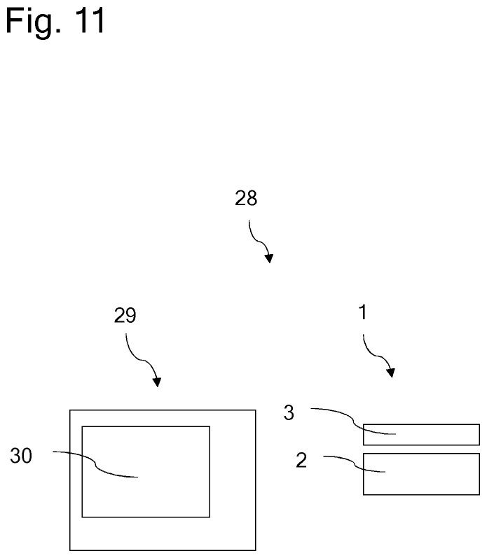

A preferred embodiment of the person monitoring system 28 according to the present invention is schematically shown in a side view in FIG. 11. The person monitoring system 28 has a person sensor system 1 according to the present invention with a person sensor 2 for measuring at least one personal parameter and with a holding device 3. In addition, the person monitoring system 28 has a person monitor 29 for outputting personal information based on the personal parameter. In this exemplary embodiment, the person monitor 29 has a display device 30, which is configured, for example, as a monitor, especially a touchscreen. Provisions may be made according to the present invention for the person monitor 29 to be configured as a mobile phone. The person sensor 2 and the person monitor 29 can preferably be coupled to each other via a wireless connection, especially via a Bluetooth connection, to transmit the at least one measured personal parameter.

While specific embodiments of the invention have been shown and described in detail to illustrate the application of the principles of the invention, it will be understood that the invention may be embodied otherwise without departing from such principles.

1. A person sensor system comprising:
a person sensor configured to determine personal parameters of a person,
a holding device configured to hold the person sensor on an article of clothing of the person in interaction with the person sensor;
a magnetic device comprising a first magnetic device section and a second magnetic device section the first magnetic device section and the second magnetic device section being configured to attract each other by magnetic force, the first magnetic device section being arranged at the person sensor and the second magnetic device section being arranged at the holding device; and
a magnetic field detection device configured to detect a magnetic field of the magnetic device, wherein the person sensor system is configured to set an operating state of the person sensor system as a function of the detected magnetic field.

2. A person sun sensor system in accordance with claim 1, wherein:
the magnetic field detection device is configured as a magnetometer for detecting a magnetic flux density of the magnetic device; and
the person sensor system is configured to control the person sensor system as a function of the magnetic flux density detected.

3. A person sensor system in accordance with claim 1, wherein:
the magnetic field detection device is configured to determine a relative position of the holding device in relation to the person sensor; or
the magnetic field detection device is configured to determine a change in position of the holding device in relation to the person sensor; or
the magnetic field detection device is configured to determine a relative position of the holding device in relation to the person sensor and to determine a change in position of the holding device in relation to the person sensor.

4. A person sensor system in accordance with claim 3, wherein:
the person sensor is configured to automatically set an operating state of the person sensor system as a function of the determined relative position; or
the person sensor is configured to automatically set an operating state of the person sensor system as a function of a change in position of the holding device in relation to the person sensor; or
the person sensor is configured to automatically set an operating state of the person sensor system as a function of the determined relative position or as a function of a change in position of the holding device in relation to the person sensor.

5. A person sensor system in accordance with claim 1, wherein the magnetic field detection device is arranged at the person sensor.

6. A person sensor system in accordance with claim 1, wherein the first magnetic device section has a magnet.

7. A person sensor system in accordance with claim 6, wherein the second magnetic device section has a ferromagnetic element.

8. A person sensor system in accordance with claim 1, wherein the second magnetic device section has a magnet.

9. A person sensor system in accordance with claim 6 wherein:
the second magnetic device section has a magnet;
the magnet of the first magnetic device section and the magnet of the second magnetic device section are arranged and oriented such that a preferred position between the person sensor and the holding device can be brought about as a result.

10. A person sensor system in accordance with claim 9, wherein the magnet of the first magnetic device section and the magnet of the second magnetic device section are arranged and oriented such that the first magnetic device section and the second magnetic device section attract each other in a first relative position from the person sensor and the holding device and repel each other in a second relative position from the person sensor and holding device, which second relative position is different from the first relative position.

11. A person sensor system in accordance with claim 10, wherein the holding device has a contact area with a first base and the person sensor has a countercontact area with a second base for arrangement at the contact area.

12. A person sensor system in accordance with claim 11, wherein the contact area has a positive-locking section and the countercontact area has a counter-positive-locking section which can be brought to mesh with the positive-locking section in the first relative position.

13. A person sensor system in accordance with claim 11, wherein:
the contact area has a shoulder relative to an adjacent section of the patient sensor or
the countercontact area has a shoulder relative to an adjacent section of the holding device; or
the contact area has a shoulder relative to an adjacent section of the patient sensor and the countercontact area has a shoulder relative to an adjacent section of the holding device.

14. A person sensor system in accordance with claim 13, wherein the shoulder has a circumferential configuration.

15. A person sensor system in accordance with claim 1, wherein the holding device is configured to be arranged at least partially in a positive-locking manner in a mount of the person sensor.

16. A person sensor system in accordance with claim 1, wherein:
the holding device has a grip surface, which projects from the holding device as well as faces away from the person sensor; and
the grip surface is configured for the manual introduction of a torque into the holding device.

17. A person sensor system in accordance with claim 1, wherein at least one partial area of the first magnetic device section is arranged in a central sensor rotation point of the person sensor and at least one partial area of the second magnetic device section is arranged in a central holding device rotation point of the holding device.

18. A person sensor system in accordance with claim 17, wherein the holding device has a holding device projection at the holding device rotation point and the person sensor has a sensor mount at the sensor rotation point, wherein the sensor mount is configured for the at least partial mounting of the holding device projection.

19. A person sensor system in accordance with claim 1, wherein:
the person sensor has at least one electrically conductive person sensor contact surface and the holding device has at least one electrically conductive holding device contact surface; wherein
the at least one person sensor contact surface as well as the at least one holding device contact surface are configured to close a common electrical circuit by direct mutual contact; and
the person sensor system is configured to control the person sensor system as a function of the state of the common electrical circuit.

20. A person sensor system in accordance with claim 19, wherein:
the common electrical circuit extends through the first magnetic device section; or
the common electrical circuit extends through the second magnetic device section; or the common electrical circuit extends through the first magnetic device section and the circuit extends through the second magnetic device section.

21. A person sensor system in accordance with claim 20, wherein:
the first magnetic device section has the person sensor contact surface or the second magnetic device section has the holding device contact surface; or
the first magnetic device section has the person sensor contact surface and the second magnetic device section has the holding device contact surface.

22. A person monitoring system for monitoring personal parameters of a person, the person monitoring system comprising:
a person sensor system with a person sensor for measuring at least one personal parameter of the person, the person sensor system comprising:
a person sensor configured to determine the at least one personal parameters of a person;
a holding device configured to hold the person sensor on an article of clothing of the person in interaction with the person sensor;
a magnetic device comprising a first magnetic device section and a second magnetic device section the first magnetic device section and the second magnetic device section being configured to attract each other by magnetic force, the first magnetic device section being arranged at the person sensor and the second magnetic device section being arranged at the holding device; and
a magnetic field detection device configured to detect a magnetic field of the magnetic device, wherein the person sensor system is configured to set an operating state of the person sensor system as a function of the detected magnetic field; and
a person monitor for outputting personal information based on the at least one personal parameter.
